(19) United States
(12) Patent Application Publication
(10) Pub. No.: US 2026/0114916 A1
(43) Pub. Date: Apr. 30, 2026

(54) DEVICES, SYSTEMS, AND METHODS FOR SEPARATING TISSUE

(71) Applicant: BOSTON SCIENTIFIC SCIMED, INC., Maple Grove, MN (US)

(72) Inventors: Gregory Todd Dalton, Austin, TX (US); Vladimir Mitelberg, Austin, TX (US); Timothy Noah Foster-Frau, Austin, TX (US); Stephen West, Austin, TX (US)

(73) Assignee: BOSTON SCIENTIFIC SCIMED, INC., Maple Grove, MN (US)

(21) Appl. No.: 19/368,414

(22) Filed: Oct. 24, 2025

Related U.S. Application Data
(60) Provisional application No. 63/711,971, filed on Oct. 25, 2024.

(51) Int. Cl. A61B 18/08; A61B 18/00

(52) U.S. Cl. CPC A61B 18/082; A61B 2018/00184; A61B 2018/00273; A61B 2018/00595; A61B 2018/0091; A61B 2018/00982

(57) ABSTRACT

A tissue-separating device, system, and method utilizing an expandable member which expands against tissue to be separated at a treatment site. The expandable member has an invertible portion extendable within and evertible out of a lumen of a tubular elongate member. An anchoring portion of the expandable member is anchored with respect to an exterior of the tubular elongate member to anchor the device with respect to the treatment site so that expansion of the expandable member against tissue does not cause rebound of the expandable member. The tubular elongate member may include one or more radially extending flow holes to inflate the anchoring portion prior to everting and expanding the invertible portion of the expandable member. A tether may be used to assist advancement and retraction of the invertible portion of the expandable member. The tether may define a lumen through which an additional device may be extended.

### CROSS-REFERENCE TO RELATED APPLICATIONS

This application claims the benefit of priority under 35 U.S.C. § 119 to U.S. Provisional Application No. 63/711,971, filed on Oct. 25, 2025, the entire disclosure of which is hereby incorporated by reference herein for all purposes.

### FIELD

The present disclosure relates generally to the field of medical devices, systems, and methods for separating anatomical tissue. More particularly, the present disclosure relates to medical devices, systems, and methods using an inflatable element to separate anatomical tissue.

### BACKGROUND

Various devices, assemblies, systems, methods, techniques, etc., exist for cutting, dissecting, resecting, excising, etc., anatomical structures (e.g., biological tissue, including lesions, etc.). The configurations of the cutting device (which may include a blade, needle, laser, or other structure capable of cutting anatomical structures) may be selected based on the particular procedure to be performed. The procedures may be performed using open surgery (accessing the interior of a patient's body by cutting open the body) or minimally invasive surgery (e.g., percutaneously, laparoscopically, endoscopically, etc.). When suspicious tissue needs to be removed, medical professionals (such as in the gastrointestinal field) may utilize techniques such as endoscopic mucosal resection (EMR) or endoscopic submucosal dissection (ESD) to remove the suspicious tissue (e.g., a lesion). For safe tissue removal, it may become necessary to access and to dissect submucosal space. Removal of suspicious tissue en bloc is preferred for pathology and containment of diseased tissue. One endoscopic procedure is third space endoscopy (also known as submucosal endoscopy), which may be performed within a space inside a wall of a gastrointestinal (GI) tract of the patient, such as in or between structurally differentiated layers of tissue (e.g., between mucosal layer and submucosal, or between submucosal and muscular layers of tissue). Third space endoscopic procedures may access deeper layers of tissue within the body (e.g., the gastrointestinal (GI) tract) by tunneling into the tissue, such as to remove lesions that are deeper than the surface of the tissue, and/or to treat conditions that are deep within the tissue (e.g., in the muscle layer of the digestive system lining). For instance, in the GI system, tunneling may be performed in the submucosal space with an endoscope, without compromising the integrity of the overlying mucosa. Typically, a fluid (e.g., water, saline, air, etc.) is injected at the treatment site to elevate/lift the tissue (to create a “bleb”) to facilitate cutting of the tissue, such as with a knife. For instance, a lifting agent may be injected into the submucosal layer to separate the mucosal layer from the muscularis layer. A sharp cutting instrument such as a blade, a knife, an electrosurgical knife, etc., may then be used to cut through the submucosa, or other tissue in third space endoscopic procedures. Typically, the cutting instrument is used to make multiple small cuts, typically sequentially, to create a desired separation of tissue gradually, such as to reduce the risk of creating too large a cut in the tissue. Separating mucosal tissue from muscularis tissue may be difficult, particularly because of the potential for various complications including, but not limited to, excessive bleeding, perforations (of the mucosa or muscularis layers of tissue), excessive tissue damage due to electrocautery burns or bruising from instruments, etc. Safer and more efficient devices, systems, methods, etc., for blunt tissue dissection using a mechanical force to separate tissue layers (in contrast with cutting tissue with a sharper instrument) and/or to address undesired bleeding during the procedure would be welcome in the field.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system for separating tissue includes a tubular elongate member having a wall defining an interior of the tubular elongate member defining a lumen, and an exterior of the tubular elongate member, and extending between a proximal end and a distalmost end of a distal end of the tubular elongate member; and an expandable member having an anchor portion extending along the exterior of the distal end of the tubular elongate member, and an invertible portion extendable within the lumen of the tubular elongate member. In some aspects, a flow hole is defined radially through the wall of the tubular elongate member proximal to the distalmost end of the tubular elongate member; and the anchor portion of the expandable member is coupled to the exterior of the tubular elongate member proximal to the flow hole whereby an inflation medium supplied into the lumen of the tubular elongate member flows radially out the flow hole to expand the anchor portion of the expandable member prior to expanding the invertible portion of the expandable member.

In some aspects, the invertible portion of the expandable member is invertible into and within the lumen of the tubular elongate member when the expandable member is in an unexpanded configuration, and contact of the expandable member with the distalmost end of the tubular elongate member retains inflation medium within the anchor portion of the expandable member so that the anchor portion may expand prior to expansion of the invertible portion within and out of the distal end of the tubular elongate member.

In some aspects, the flow hole is configured to assist with removal of inflation medium from the anchoring portion of the expandable member to fully deflate the expandable member.

In some aspects, the system further includes a tether operably coupled with the invertible portion of the expandable member to assist with retracting the expandable member into the lumen of the tubular elongate member. In some aspects, the tether defines a lumen therethrough, the system further comprising an additional medical device insertable through the lumen through the tether. In some aspects, the additional medical device is a cauterization device. In some aspects, the additional medical device is an electrocauterization wire, the system further comprising a protective sleeve surrounding the electrocauterization wire to protect a portion of the invertible portion of the expandable member within the lumen of the tubular elongate member from the electrocauterization device.

In some aspects, the system further includes an additional medical device extending along the exterior of the tubular elongate member or within an additional device lumen defined through the tubular elongate member separate from the lumen of the tubular elongate member. In some aspects, the additional medical device has a cauterization tip extendable distal to the distal end of the tubular elongate member.

In some aspects, the system further includes a control handle operably coupled with the proximal end of the tubular elongate member, the control handle having an inflation port fluidly communicated with the lumen of the tubular elongate member and a fluid supply to supply inflation medium distally into the lumen of the tubular elongate member to extend radially through the flow hole to inflate the anchor portion of the expandable member prior to inflating the invertible portion of the expandable member.

In accordance with various principles of the present disclosure, a device for separating tissue at a treatment site has an end effector including a tubular elongate member defined by a wall defining an interior of the tubular elongate member defining a lumen, and an exterior of the tubular elongate member, and extending between a proximal end and a distalmost end of a distal end of the tubular elongate member; and an expandable member having an anchor portion extending along the exterior of the distal end of the tubular elongate member to an anchoring location proximal to the distalmost end of the tubular elongate member, and an invertible portion extendable within the lumen of the tubular elongate member. In some aspects, the anchor portion of the expandable member is inflatable to anchor the tissue-separating end effector with respect to the treatment site; and the invertible portion is inflatable to extend distally out of the lumen of the tubular elongate member after inflation of the anchor portion.

In some aspects, a flow hole is defined radially through the wall of the tubular elongate member proximal to the distalmost end of the tubular elongate member and fluidly communicated with the anchor portion of the expandable member. In some aspects, the anchoring location is proximal to the flow hole, whereby an inflation medium supplied into the lumen of the tubular elongate member flows radially out the flow hole to expand the anchor portion of the expandable member without expanding the invertible portion of the expandable member. In some aspects, the flow hole is configured to assist with removal of inflation medium from the anchoring portion of the expandable member to fully deflate the expandable member.

In some aspects, the tissue-separating end effector further includes a tether operably coupled with the invertible portion of the expandable member to assist with retracting the expandable member into the lumen of the tubular elongate member.

In accordance with various principles of the present disclosure, a method of separating tissue at a treatment site includes extending a tissue-separating end effector to the treatment site, the tissue-separating end effector comprising a tubular elongate member defining a lumen therethrough, and an expandable member, the expandable member having an anchoring portion coupled to an anchoring location along exterior of the tubular elongate member proximal to a distalmost end of the tubular elongate member, and an invertible portion extendable within the lumen of the tubular elongate member; expanding the anchoring portion of the expandable member prior to expanding the invertible portion of the expandable member to anchor the tissue-separating end effector with respect to the treatment site; and after anchoring the tissue-separating end effector with respect to the treatment site, expanding the expandable member to evert distally out of the tubular elongate member to separate tissue.

In some aspects, the method further includes inflating the anchoring portion with an inflation medium to expand the anchoring portion. In some aspects, a flow hole is defined radially through a wall defining the distal end of the tubular elongate member and proximal to the anchoring location, the method further comprising delivering an inflation medium into the lumen of the flexible elongate member to extend radially through the flow hole and into the anchoring portion of the expandable member to inflate the anchoring portion of the expandable member.

In some aspects, the method further includes assisting eversion or inversion of the invertible portion of the expandable member by axial movement of a tether extending through the lumen of the tubular elongate member and operably coupled with the invertible portion of the expandable member. In some aspects, the method further includes extending an additional medical device through a lumen defined through the tether.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not necessarily drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a system for separating anatomical tissue formed in accordance with aspects of the present disclosure.

FIG. 2 illustrates a cross-sectional view along line II-II of FIG. 1.

FIGS. 3A-3D illustrate various configurations of an expandable member of a system such as illustrated in FIG. 1.

FIG. 4 illustrates an example of an embodiment of a system for separating anatomical tissue formed in accordance with aspects of the present disclosure and including an additional treatment device.

FIG. 5 illustrates an example of an embodiment of a system for separating anatomical tissue formed in accordance with aspects of the present disclosure and including an additional treatment device.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

Various devices, systems, and methods utilize a tissue-separating end effector having an expandable member which is actuated to separate and/or tunnel into anatomical tissue. It will be appreciated that anatomical tissue may alternatively be referenced as biological tissue, tissue of a patient, anatomical structure, etc., without intent to limit, and, for the sake of convenience, referenced herein is made simply to “tissue”. For the sake of convenience, the tissue contacted by the expandable member is referenced herein as “contacted tissue”.

In some aspects, the expandable member is actuated by being expanded with respect to another component of the tissue-separating system to effect separation of tissue and/or tunnelling into tissue contacted by the moving expandable member. In accordance with various principles of the present disclosure, the tissue-separating end effector has an anchoring configuration in which a portion of the tissue-separating end effector is configured to be anchored with respect to tissue. The expandable member may thus be actuated and moved to separate tissue without the movement of the expandable member causing undesired or inadvertent movement of the tissue-separating end effector as the expandable member expands and pushes against tissue. Actuation of an expandable member thus causes movement in the desired direction without the potential of the expandable member pushing off tissue and causing reverse movement of the tissue-separating end effector (rebound retraction, reversion, etc., opposite the direction in which the tissue-separating end effector is to be advanced to separate tissue).

In some aspects, the expandable member is coupled with a tubular elongate member and inverted into the lumen of the tubular elongate member. Typically, the expandable member is in an unexpanded and/or collapsed configuration when inverted within the lumen of the tubular elongate member. Upon expansion of the expandable member (e.g., with an inflation fluid), the expandable member everts out of the lumen of the tubular elongate member. As the expandable member is everted and is contacted with anatomical tissue, the expandable member moves distally from the tubular elongate member and laterally away from the longitudinal axis of the tubular elongate member and separates the contacted tissue. In accordance with various principles of the present disclosure, an anchoring portion of the tissue-separating end effector anchors the tissue-separating end effector with respect to tissue so that distal advancement of the expandable member does not cause rebound and reverse movement of the tissue-separating end effector. In some aspects, the anchoring portion is a portion of the expandable member.

In some aspects, the expandable member has an external end coupled to (e.g., surrounding, encircling, etc.) the exterior of a tubular elongate member, and extends into the tubular elongate member (e.g., wrapping over the distalmost end of the tubular elongate member and into the lumen of the tubular elongate member) to be stored/stowed therein for insertion into a patient and delivery to a treatment site. The expandable member thus has an internal portion within the tubular elongate member, and an external portion outside and surrounding the distal end of the tubular elongate member. In some aspects, one or more flow holes are defined through a portion of the wall defining the distal end of the tubular elongate member over which the external portion of the expandable member extends. The flow holes fluidly communicate the lumen defined within the tubular elongate member with the external portion of the expandable member. The external portion of the expandable member may thus be readily expanded, despite a degree of sealing which may result from the expandable member wrapping around and contacting the distalmost end of the tubular elongate member. In some aspects, inflation medium is directed to the external portion of the expandable member through the flow holes to cause expansion of the external portion of the expandable member prior to expansion of other portions of the expandable member. For instance, the inflation medium may be directed to the external portion of the expandable member without passing through intermediate portions of the expandable member, and bypassing a seal created along the distalmost end of the tubular elongate member. Expansion of the external portion of the expandable member before eversion of the internal portion of the expandable member may anchor the expandable member to allow the further expansion of the expandable member to separate tissue with little to no rebounding from the contacted tissue. The expansion of the external portion of the expandable member may facilitate expansion of the expandable member by distributing inflation medium to portions of the expandable member furthest from the inflation medium source without passing through intermediate portions of the expandable member, such as the inverted portion of the expandable member positioned within the lumen of the tubular elongate member. Inflation of the external portion of the expandable member may serve to withdraw and evert the inverted portion of the expandable member from the lumen of the expandable member. In some aspects, because the external portion of the expandable member presents a greater surface area than the inverted portion, less pressure is used to inflate such section of the expandable member, facilitating filling of the expandable member with inflation medium to expand the expandable member.

In some aspects, devices, systems, and method described herein may be used for performing submucosal medical procedures in a desired area of the digestive tract using an endoscope. More particularly, devices, systems, and method described herein may be used for various endoscopic procedures. For instance, devices described herein may be used, without limitation, as a submucosal tunneling instrument, a submucosal dissection instrument, a mucosal resection device.

In some aspects, devices and systems described herein may be used to perform “third space” procedures and/or procedures in other transluminal spaces of a patient which are generally difficult to access with current devices. Typically, a small incision is made (e.g., a with a cutting instrument) for accessing the “third space”, and a blunt medical instrument is inserted through the incision. It will be appreciated that terms such as medical instrument, tool, device, accessory, etc., may be used interchangeably herein without intent to limit. In some aspects, a fluid (e.g., water, saline, air, etc.) is injected into target tissue/the treatment site to elevate/lift the tissue (to create a “bleb”) to facilitate cutting of the tissue and/or insertion of the tissue separating/tunnelling device. The tissue separating/tunnelling device may then be inserted to accessing deep into the tissue (e.g., in the submucosal space or beyond) without compromising the integrity of the overlying tissue (e.g., the mucosa). Examples of third space procedures include, but are not limited to, per oral endoscopic myotomy (POEM), endoscopic submucosal dissection (ESD), Zenker's diverticulotomy (ZPOEM), endoscopic gastric pyloromyotomy (GPOEM), submucosal tunneling endoscopic resection (STER), and tunneling recanalization of the esophagus (POETRE), among others.

In some aspects, devices, systems, and methods described herein may include a device for addressing any inadvertent bleeding which may occur during a procedure using a device or system as described herein. For instance, devices and systems described herein may include a device capable of controlling and/or stopping inadvertent bleeding, such as a cauterization/electrocauterization device.

In some aspects, multiple tunnels may be formed in tissue by a device, system, or method in accordance with various principles of the present disclosure. In some aspects, various strands of biological materials, such as strands of tissue, connective tissue, blood vessels, etc., may be cut to connect the various tunnels to form a larger space and/or to facilitate separation of the overlying tissue from the underlying tissue.

Various embodiments of devices, systems, and methods usable to separate and/or tunnel into tissue will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiments are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified herein and in the drawings by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element).

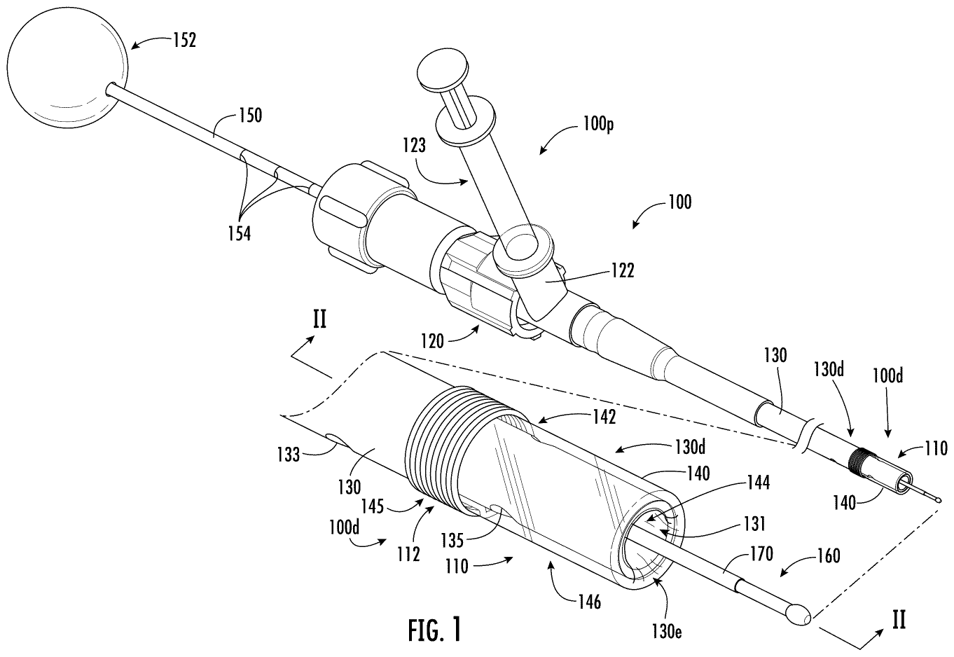

Referring to the drawings, an example of an embodiment of a system 100 formed in accordance with various principles of the present disclosure is illustrated in FIG. 1. The illustrated system 100 is configured to separate and/or tunnel into tissue. For the sake of convenience, and without intent to limit, reference is simply made to separating tissue, and the system 100 is referenced herein as a tissue-separating system 100. The tissue separating system 100 has a distal tissue-separating end 100d with a tissue-separating end effector 110, a proximal control end 100p with a control handle 120, and a tubular elongate member 130 (e.g., catheter, sheath, etc.) extending between the distal tissue-separating end 100d and the proximal control end 100p. In some aspects, at least a portion of the tissue-separating system 100 is sufficiently flexible to be navigated within and through tortuous passages within a patient's body, such as to perform a transluminal, transcatheter, endoluminal, etc., procedure with the tissue-separating end effector 110. For instance, at least a distal portion of the tubular elongate member 130 and at least elements within at least the distal portion 130d of the tubular elongate member 130 may be sufficiently flexible to be inserted into a patient's body and navigated within and through curved (e.g., tortuous) passages within a patient's body. In some aspects, the tissue-separating system 100 may be used with a medical scope, and may be extended through a working channel of a medical scope, such as in a manner known to those of ordinary skill in the art.

In accordance with various principles of the present disclosure, the tissue-separating end effector 110 includes an expandable member 140, such as a balloon. The expandable member 140 is formed of a flexible material capable of being filled with an inflation medium (e.g., saline, air, etc.) to expand. The flexible material may be non-compliant, semi-compliant, or compliant, such as depending on the tissue to be separated. For instance, the expandable member 140 may be formed from a biocompatible polymeric material such as a polyester, polyethylene terephthalate (PET), olefins, elastomers, thermoplastic elastomers, vinyls, polyamides, polyimides, fluoropolymers, and/or copolymers and/or blends thereof, etc. A portion of the expandable member 140 is contacted with tissue (e.g., advanced into contact with tissue) and causes separation of the tissue as the expandable member 140 expands. In some aspects, tangential movement of the material of the expandable member 140 with respect to the tissue, as the expandable member 140 expands, pulls on the tissue to cause separation of the tissue. It will be appreciated that terms such as separating, snapping, breaking, tunnelling, dissecting, resecting, etc., and other grammatical forms thereof, may be used interchangeably herein, and without intent to limit, to describe the affect the expandable member 140 has on tissue.

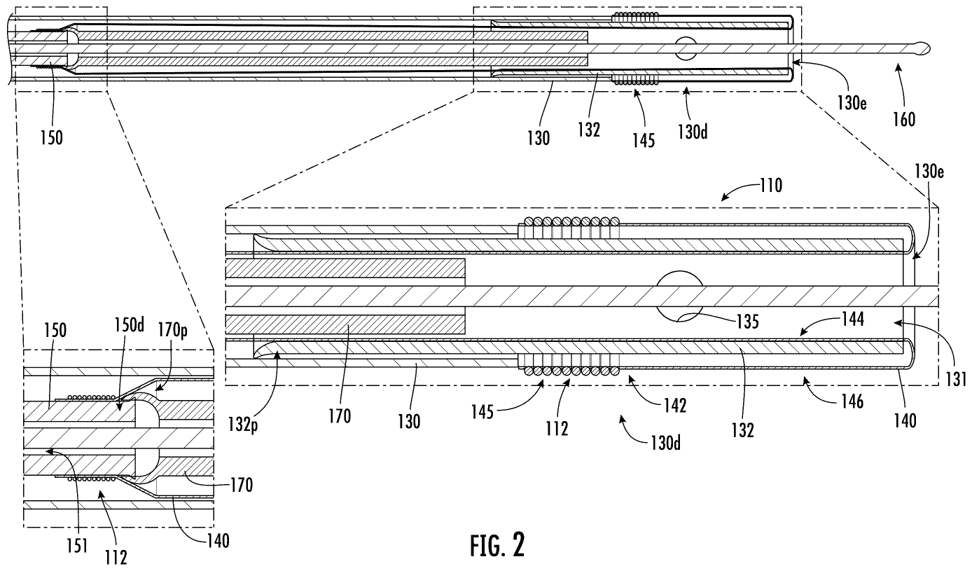

In some aspects, a portion of the expandable member 140 extends over the distal end 130d of the tubular elongate member 130 and a portion of the expandable member 140 is configured to be inverted into or stored or stowed (such terms being usable interchangeably herein without intent to limit unless stated) within the lumen 131 of the tubular elongate member 130, as illustrated in FIG. 2, showing cross-sectional view along line II-II of FIG. 1. For instance, the tissue-separating end effector 110 may be advanced and delivered to a target site in a delivery configuration with the expandable member 140 inverted and stowed within the lumen 131 of the tubular elongate member 130. In some aspects, the expandable member 140 defines a periphery 142 (e.g., circumference, boundary, edge, etc.) which is coupled to the outer tubular elongate member 130 (e.g., along the exterior/outer surface of the distal end 130d of the outer tubular elongate member 130), and a radially-inward portion 144 (e.g., a portion along a generally central region, inwardly spaced from the periphery 142) which may be retractable within the lumen 131 of the outer tubular elongate member 130. For instance, the periphery 142 of the expandable member 140 may be affixed to the distalmost end and/or the exterior of the outer tubular elongate member 130 in any of a variety of manners, such as mechanical affixing (such as with fixing bands or string 112 extending circumferentially around the exterior of the periphery 142 of the expandable member 140 and the exterior of the tubular elongate member 130, a crimping ring, etc.); and/or bonding such as with adhesive; and/or joining using heat such as by welding, etc., the present disclosure not being limited in this regard. In some aspects, as may be appreciated with reference to FIG. 2, the distal end 100d of the tubular elongate member 130 includes a separately formed tip insert 132 coupled (e.g., bonded, welded, adhered, etc.) to the main body of the tubular elongate member 130. The tip insert 132 may have a smaller outer diameter than the tubular elongate member 130, thereby reducing the outer diameter of the tissue-separating end effector 110 the longitudinal location at which the expandable member 140 is extended over the exterior of the tip insert 132 (in contrast with a larger outer diameter which would result if the expandable member 140 were extended over the larger outer diameter of the exterior of the tubular elongate member 130). Optionally, an aperture 133 may be provided in the tubular elongate member 130 to facilitate coupling tip insert 132 with the distal end 130d of the tubular elongate member 130. For the sake of convenience, and without intent to limit, references to the distal end 130d and/or distalmost end 130e of the tubular elongate member 130 are to be understood as references to the distal end 130d and/or distalmost end 130e of the tip insert 132, if provided, or to the distal end 130d and/or distalmost end 130e of the main body of the tubular elongate member 130 if the tip insert 132 is not provided. In some aspects, the radially-inward portion 144 of the expandable member 140 is folded, creased, crumpled, or otherwise compacted into the lumen 131 of the tubular elongate member 130. It will be appreciated that in the everted configuration of the expandable member 140, the periphery 142 remains outside, along the exterior of the tubular elongate member 130, whereas the radially-inward portion 144 is stowed within the lumen 131 of the tubular elongate member 130. It will further be appreciated that in the expanded configuration of the expandable member 140, the periphery 142 may be considered the proximal end 140p of the expandable member 140 and the everted radially-inward portion 144 may be considered the distal end 140d of the expandable member 140.

The expandable member 140 may be caused to expand by filling the lumen 131 of the tubular elongate member 130 with an inflation medium. In the example of an embodiment of a tissue-separating system 100 illustrated in FIG. 1, the lumen 131 of the tubular elongate member 130 is fluidly communicated/coupled with an inflation port 122 formed on or along (e.g., fluidly coupled with) the control handle 130. It will be appreciated that reference may be made to fluidly communicated and fluidly coupled interchangeably herein without intent to limit unless specifically indicated. The inflation port 122, in turn, is fluidly coupled with a fluid supply 123. The example of an embodiment of a fluid supply 123 illustrated in FIG. 1 is in the form of a fluid-filled syringe, but may be in any other appropriate/desired form known to those of ordinary skill in the art. The fluid supply 123 is operable to supply an inflation medium via the inflation port 122 to the lumen 131 of the tubular elongate member 130 to cause expansion of the expandable member 140 along with eversion of the expandable member 140 out of the distal end 130d of the tubular elongate member 130. For instance, the inflation medium impacts the everted portion of the expandable member 140 within the lumen 131 of the tubular elongate member 130 and pushes the expandable member 140 distally out of the lumen 131. As may be appreciated, the supply of inflation medium may be controlled, as desired, to control inflation or deflation of the expandable member 140, such as medically indicated by the procedure, as may be appreciated by those of ordinary skill in the art. For instance, careful maintenance and/or control of inflation medium pressure, and/or balancing of pressure within the lumen 131 and within the fluid supply 123, allows for precise control of expansion and retraction of the expandable member 140. The expandable member 140 may thus be extended out of the tubular elongate member 130 with precise control to extend a precise length of the expandable member 140 distally out of the tubular elongate member 130 and into tissue. Control of back pressure, such as from the fluid supply 123 on the lumen 131 of the tubular elongate member 130, facilitates expansion as well as full return, inversion, and/or storage of the expandable member 140 within the lumen 131.

Optionally, a tether 150 is coupled to the expandable member 140, and extends proximally to the control handle 130 to provide additional control of expansion or retraction of the expandable member 140. The tether 150 may be an elongate member, such as, without limitation, a tubular element, a pull wire, a cord, a string, a filament, etc., capable of pulling the expandable member 140 proximally and/or pushing the expandable member 140 distally to control inversion (and storage) or eversion (and extension) of the expandable member 140 with respect to the tubular elongate member 130. In some aspects, the tether 150 is an elongate tubular member with a lumen therethrough, as discussed in further detail below. In some aspects, the tether 150 is coupled with respect to a radially-inward portion 144 of the expandable member 140, such as illustrated in FIG. 2. In some aspects, the radially-inward portion 144 of the expandable member 140 is affixed over an exterior surface of the distal end 150d of the tether 150, as illustrated in FIG. 2. Various manners of coupling the expandable member 140 and tether 150 include, without limitation, mechanical affixing (such as with fixing bands or string 112 extending circumferentially around the exterior of the expandable member 140 and the distal end 130d of the tether 150, a crimping ring, etc.); and/or bonding such as with adhesive; and/or joining using heat such as by welding, etc., the present disclosure not being limited in this regard. Optionally, the distal end 150d of the tether 150 may be outwardly flared to abut an outwardly flared proximal end 132p of the tip insert 132, if provided. Such configuration may limit the distal travel of the tether 150 and may be helpful in avoiding overpressurizing the expandable member 140.

A pull handle 152 may be provided on or along the proximal end 150p of the tether 150 to facilitate grasping and control of movement of the tether 150 (e.g., manual control by a medical professional). For instance, pulling proximally on the tether 150 pulls the expandable member 140 member proximally. In some aspects, the tether 150 may assist with inversion and return of the expandable member 140 back into the lumen 131 of the tubular elongate member 130 after use of the expandable member 140 in an expanded configuration. For instance, although release/suctioning/removal of the inflation medium may cause collapse of an expandable member 140 which has been inflated with such inflation medium, further inversion and retraction of the expandable member 140 into the tubular elongate member 130 may be assisted by pulling proximally on the tether 150. Indicia 154 may be provided along a proximal region of the tether 150 (along the proximal end 150p of the tether 150 but distal to the pull handle 152) to indicate the length of deployment and/or retraction of the expandable member 140, such as for verification by a medical professional. Further details and features and uses of the tether 150 are described in further detail below.

Eversion of the expandable member 140, such as upon pushing the tether 150 and/or filling of the lumen 131 of the tubular elongate member 130 with inflation medium, causes the radially-inward portion 144 of the expandable member 140 stowed within the lumen 131 of the tubular elongate member 130 to be pushed distally out of the tubular elongate member 130 to expand the expandable member 140 out of the tubular elongate member 130. As noted above, expansion of the expandable member 140 outwardly from the tubular elongate member 130 and against tissue to be separated by the tissue-separating end effector 110 may cause the expandable member 140 to impact the tissue without sufficient shear force to tunnel into the tissue. In prior balloon tunnelling devices, such impact against tissue may cause the expandable balloon member to rebound against the tissue, thus moving the balloon-tunnelling device proximally away from the tissue to be separated, backing the device and system out of the tissue to be separated. In accordance with various principles of the present disclosure, a portion of the tissue-separating end effector 110 is configured to be anchored with respect to tissue (e.g., tissue proximal to target tissue at the treatment site) prior to expansion of the expandable member 140 out of the tubular elongate member 130. Such anchoring inhibits and/or prevents rebounding of the expandable member 140 against tissue as the expandable member 140 expands against tissue. In some aspects, the anchoring of the tissue-separating end effector 110 creates a secure point of attachment to allow the expandable member 140 to expand and evert out of the tubular elongate member 130 and move with respect to tissue to separate the tissue without pushing off and rebounding from the tissue.

In some aspects, anchoring of the tissue-separating end effector 110 of the example of an embodiment illustrated in FIG. 1 is achieved by anchoring the distal end 130d of the tubular elongate member 130 with respect to tissue, such as by expanding a limited portion of the expandable member 140 extending over the exterior of the distal end 130d of the tubular elongate member 130. Such external portion of the expandable member 140 may be referenced as an anchoring portion 146. In accordance with various principles of the present disclosure, at least one flow hole 135 is provided radially through the wall defining the distal end 130d of the tubular elongate member 130 proximal to the terminal, distalmost end 130e of the tubular elongate member 130. The flow hole 135 is fluidly communicated with the anchoring portion 146 of the expandable member 140 to facilitate expansion of an anchoring portion 146 outside the tubular elongate member 130. In the example of an embodiment illustrated in FIG. 1 and FIG. 2, the periphery 142 of the expandable member 140 is coupled to the exterior of the wall of the tubular elongate member 130 at an anchoring location 145 proximal to the flow hole 135 in a fluid-tight manner to define an anchoring portion 146 of the expandable member 140 between the anchoring location 145 and the distalmost end 130e of the tubular elongate member 130. The longitudinal extent of the anchoring portion 146 between the anchoring location 143 and the distalmost end 130e of the tubular elongate member 130 is not coupled to the exterior of the tubular elongate member 130. Thus, inflation medium flowing through one or more flow holes 135 inflates the anchoring portion 146 to anchor the distal end 130d of the tubular elongate member 130 with respect to tissue at the treatment site.

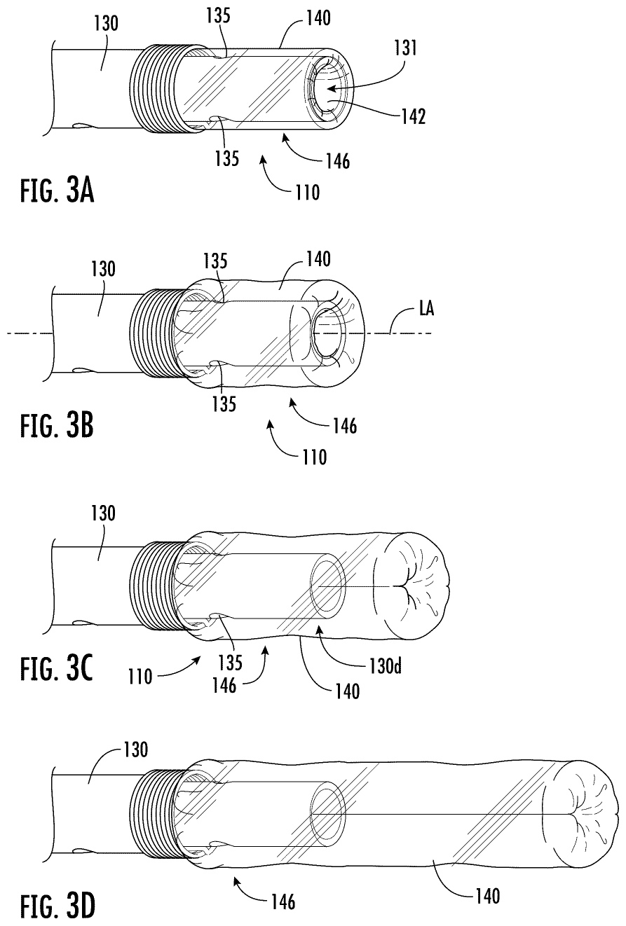

Inflation of the anchoring portion 146 of the expandable member 140 to anchor the tissue-separating end effector 110 of a system 100 such as illustrated in FIG. 1 may be further appreciated with reference to FIG. 3A, FIG. 3B, FIG. 3C, and FIG. 3D. The tissue-separating end effector 110 may be extended to a treatment site in a configuration such as illustrated in FIG. 3A, with the expandable member 140, including the anchoring portion 146 thereof, in a generally compact, generally collapsed configuration. In accordance with various principles of the present disclosure, once the tissue-separating end effector 110 has been extended to a treatment site at which target tissue (e.g., tissue to be removed) is located, the anchoring portion 146 may be expanded with inflation medium prior to eversion and expansion of the expandable member 140 to anchor the tissue-separating end effector 110 with respect to the treatment site. The inflation medium is supplied axially through the lumen 131 of the tubular elongate member 130 and radially through the one or more flow holes 135. As the lumen 131 of the tubular elongate member 130 is supplied with inflation medium, the inflation medium exits the tubular elongate member 130 laterally/radially (with respect to the longitudinal axis LA) through at least one flow hole 135 and into the anchoring portion 146. It will be appreciated that the flow hole 135 provides a path of less resistance than further longitudinal distal movement to push the radially-inward portion 144 of the expandable member 140 out of the lumen 131. As illustrated in FIG. 3B, the inflation medium inflates and radially expands the anchoring portion 146 of the expandable member 140 laterally away from the longitudinal axis LA of the tissue-separating end effector 110. In some aspects, the tissue-separating end effector 110 is extended into a small incision at the treatment site (e.g., an elevated portion or “bleb”), and radial expansion of the anchoring portion 146 enlarges the diameter of the tissue-separating end effector 110 sufficiently to resist being backed out of the incision upon expansion of the expandable member 140 against tissue at the treatment site to separate the tissue. In some aspects, the tether 150 (e.g., as illustrated in FIG. 2) may be used to restrain the inverted portion of the expandable member 140 within the tubular elongate member 130 from everting and expanding distally out of the tubular elongate member 130. For instance, a medical professional may prevent distal movement of the tether 150 which may otherwise occur upon inflation medium impacting and distally pushing the inverted portion of the expandable member 140 to which the tether 150 is coupled. In some aspects, the medical professional may hold the pull handle 152 (e.g., as illustrated in FIG. 1), such as to prevent distal movement of the tether 150 and/or to maintain tension on the tether 150. The anchoring portion 146 thus may be inflated prior to expansion of the expandable member 140 distally out of the tubular elongate member 130. The expanded anchoring portion 146 may be expanded into contact with surrounding tissue to anchor the tissue-separating end effector 110 with respect to the surrounding contacted tissue and with respect to the treatment site.

With the tissue-separating end effector 110 thus anchored with respect to tissue, continued expansion of the inverted portion of the expandable member 140 distally out of the tubular elongate member 130 may be permitted, as illustrated in FIG. 3C. For instance, the medical professional may release the hold on the tether 150 to allow the inflation medium to push the expandable member 140 out of the distal end 130d of the tubular elongate member 130 and to evert.

In some aspects, the provision of one or more flow holes 135 radially through the wall of the tubular elongate member 130 allows the expandable member 140 to be deployed with less fluid pressure, and/or allows the medical professional greater control over expansion of the expandable member 140. As may be appreciated, the wrapping of the expandable member 140 over the distalmost end 130e between the external portion of the expandable member 140 and the inverted portion of the expandable member 140 within the lumen 131 of the tubular elongate member 130 may create a sealing effect between the portion of the expandable member 140 external to the tubular elongate member 130 and the portion of the expandable member 140 within the lumen 131 of the tubular elongate member 130. The ability of inflation medium to flow through the flow holes 135 allows inflation medium to fill the external, anchoring portion 146 of the expandable member 140 quicker than by flowing out the distal end 130d of the tubular elongate member 130 to the anchoring portion 146. Moreover, such flow path bypasses the portion of the expandable member 140 contacting (and substantially sealing against) the distalmost end 130e of the tubular elongate member 130 and which would resist distal flow of inflation medium out of the tubular elongate member 130. In some aspects, because the anchoring portion 146 of the expandable member 140 presents a greater surface area to the inflation medium, less pressure is needed to inflate the anchoring portion 146. Moreover, because the external and internal portions of the expandable member 140 may be inflated despite any sealing along the distal end of the tubular elongate member 130, the expandable member 140 may be inflated faster, and in a more controlled manner than if the internal portion were expanded first, to overcome the sealing effect at the distal end 130d of the tubular elongate member 130 and then to expand the external portion of the expandable member 140.

As the inverted portion of the expandable member 140 continues to evert and expand distally from the tubular elongate member 130, the anchoring portion 146 of the expandable member 140 maintains the position of the tissue-separating end effector 110 relative to the treatment site so the intended procedure may be performed with the tissue-separating end effector 110. Anchoring of the anchoring portion 146 of the expandable member 140, such as described above, creates a secure point of attachment for the tissue-separating end effector 110 to resist the reactionary force of the tissue generated by expansion of the expandable member 140 against the tissue, and thereby to prevent the tissue-separating end effector 110 from being backed out while separating or tunneling into the targeted tissue. The medical professional may thus apply less, if any, force to resist backing out of the catheter as the tissue-separating end effector 110 impacts tissue to separate the tissue. Distal advancement, eversion, and/or expansion of the expandable member 140 with respect to tissue thus separates the tissue, such as by shearing forces of the expandable member 140 with respect to the tissue. The expandable member 140 may be expanded to its full extent, such as illustrated in FIG. 3D, with the anchoring portion 146 ensuring that the expandable member 140 expands in the intended direction and manner, rather than rebounding, sliding, shifting, etc., undesirably and/or unpredictably. As may be appreciated, anchoring the tissue-separating end effector 110, such as with the anchoring portion 146 as described, stabilizes the tissue-separating end effector 110 during use, and allows for a more stable and controlled operation of the expandable member 140, improved control of the fluid supply 123 and/or the pull handle 152 of the tether 150, more controlled and precise tissue separation, and overall improved control of the tissue-separating system 100. This level of control is beneficial if not crucial for safe and effective tissue separation, such as in EMR or ESD procedures.

As noted above, to retract the expandable member 140, the inflation medium may be removed (e.g., suctioned out of) the tubular elongate member 130 to deflate or collapse the expandable member 140. Optionally, the tether 150 may be pulled proximally to assist with inversion of the expandable member 140 proximally into the lumen 131 of the tubular elongate member 130. The one or more flow holes 135 may assist with removal of the inflation medium to collapse the expandable member 140. For instance, without the outlet provided by the flow holes 133, inflation medium could become trapped between the exterior of the tubular elongate member 130 and the portion of the expandable member 140 extending along the exterior of the tubular elongate member 130. The expandable member 140 may thereby be retracted with less force than required by prior devices which do not have the flow holes 135 described herein.

As noted above, the tether 150 may be advantageously used to control movement of the expandable member 140, such as by moving the pull handle 152 distally to move the expandable member 140 distally (such as in conjunction with and assisting the inflation medium), or proximally pulling the pull handle 152 to retract the expandable member 140 back into the tubular elongate member 130 The use of the tether 150 may thus provide increased precision in expansion and/or retraction of the expandable member 140 to control the use of the expandable member 140 in separating tissue. In some aspects, slack linkage is provided to remove tension, such as between the expandable member 140 and the tether 150, by incorporating a full or partial section of slack within the tether 150. The medical professional thereby has improved and increased control of the length and speed of deployment of the expandable member 140 from the tubular elongate member 130. The slack linkage may also relieve tension on the expandable member 140 during storage within the tubular elongate member 130. For instance, if the tubular elongate member 130 is curved, then the radial outermost portion of the tubular elongate member 130 may be under a degree of tension, and the radial innermost portion of the tubular elongate member 130 may be somewhat compressed, with the tether 150 and/or expandable member 140 therein either compressed or under tension. Such tension on the components of the system 100 could potentially affect the performance or longevity of the expandable member 140. Slack linkage as described herein reduces/eliminates tension on the expandable member 140 during storage. The slack linkage may allow for adjustment of tension or compression of the tether 150 so that the tether 150 is neither under tension nor compressed. Additionally or alternatively, by removing tension on the tether 150, the length and speed of deployment of the expandable member 140 may be controlled with more precision. Such improved control facilitates delicate procedures such as tissue separation and dissection. As may be appreciated, the slack linkage works in conjunction with other control mechanisms of the system 100, such as the pull handle 152 and pressure valves associated with the fluid supply 123, to provide a comprehensive system for precise manipulation of the expandable member 140. The system 100 thus allows medical professionals to deploy the expandable member 140 to a specific length, retract the expandable member 140 as needed, and maintain better overall control during procedures.

In some aspects, as noted above, the tether 150 may be in the form of a tubular element. The tether 150 thus may include a lumen 151 therethrough which may allow passage of additional devices through the tether 150. For instance, it may be advantageous to have a device available as the tissue-separating end effector 110 is separating tissue in case a blood vessel is inadvertently snapped, or other bleeding occurs, and/or a device which may cut away or otherwise eliminate tissue remaining between tunnels created in tissue by the tissue-separating end effector 110. In the example of an embodiment of a system 100 illustrated in FIG. 1 and FIG. 2, an additional device 160 is extended distally through the lumen 151 of the tether 150. The additional device 160 is illustrated as a cauterization device (e.g., an electrocautery device such as beaded electrocautery wire). However, the present disclosure is not limited in this regard. For instance, as also noted above, the tissue-separating end effector 110 may be used to tunnel through tissue, but may leave sections of surrounding tissue or strands uncut. A cutting instrument (the cauterization device or a sharper cutting instrument, such as a cutting knife) may be inserted through the lumen 151 of the tether 150 to cut the remaining tissue at the treatment site and thereby to connect the tunnels until the dissection of target tissue at the treatment site is complete. The tissue above the cut area may then be removed if desired or medically indicated. A protective sleeve 170 may be provided between the expandable member 140 and the additional device 160, such as surrounding the additional device 160. For instance, the protective sleeve 170 may be configured to protect the expandable member 140 from a cauterization device, such as from heat generated by the cauterization device. It will be appreciated that any of a variety of devices, such as a Tuohy-Borst connector, may be used to allow passage of an additional device 160 through the tether 150 while an inflation medium fills the space between the interior of the tubular elongate member 130 and the exterior of the tether 150 to expand the expandable member 140.

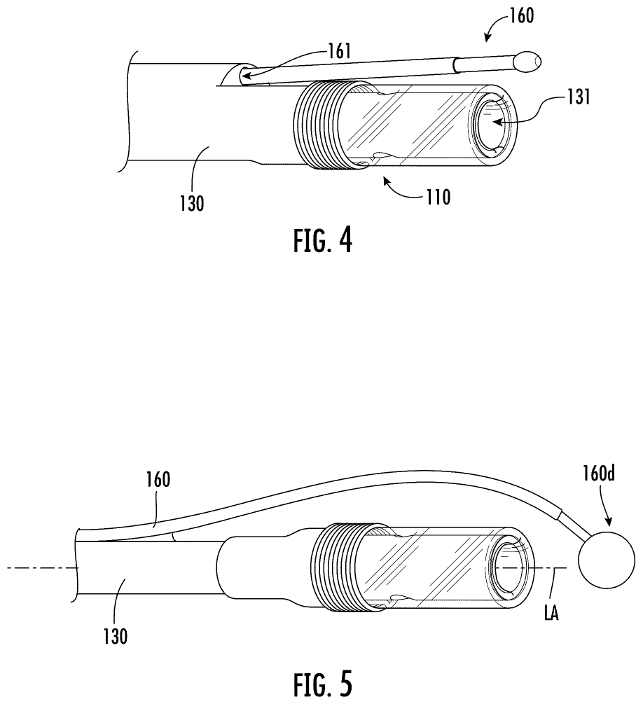

Instead of delivering an additional device 160 through the lumen 151 of the tether 150 and the lumen 131 of the tubular elongate member 130, the additional device 160 may, instead, be delivered outside (e.g., along the exterior of) the lumen 131 of the tubular elongate member 130. For instance, the tubular elongate member 130 may be a dual lumen tubular member, with an additional lumen 161 extending along the lumen 131 to deliver the additional device 160 along the exterior of the tubular elongate member 130 and the expandable member 140, such as illustrated in FIG. 4. In some aspects, the outlet of additional lumen 161 may be proximal to the anchoring portion 146 of the expandable member 140 along the longitudinal extent of the tissue-separating end effector 110. In some aspects, the distal end 160d of the additional device 160 is distally extendable distal to the distal end 130d of the tubular elongate member 130. In some aspects, the additional device 160 may be delivered alongside the tubular elongate member 130 as a separate element, such as illustrated in FIG. 5. In some aspects, such as illustrated in FIG. 5, the additional device 160 may be curved along the longitudinal axis LA of the tissue-separating end effector 110. In such embodiment, although the additional device 160 extends along the exterior of the tubular elongate member 130, radially outwardly from the longitudinal axis LA of the tissue-separating end effector 110, the distal end 160d is closer to the longitudinal axis LA of the tissue-separating end effector 110. In some aspects, the distal end 160d of the example of an embodiment illustrated in FIG. 5 extends distal to the distal end 130d of the tubular elongate member 130.

In view of the above, it should be understood that the various embodiments illustrated in the figures have several separate and independent features, which each, at least alone, has unique benefits which are desirable for, yet not critical to, the presently disclosed devices and systems and methods. It is to be understood by one of ordinary skill in the art that the present discussion is a description of illustrative examples of embodiments only, and is not intended as limiting the broader aspects of the present disclosure.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for separating tissue, said device comprising:
a tubular elongate member having a wall defining an interior of said tubular elongate member defining a lumen, and an exterior of said tubular elongate member, and extending between a proximal end and a distalmost end of a distal end of said tubular elongate member; and
an expandable member having an anchor portion extending along the exterior of the distal end of said tubular elongate member, and an invertible portion extendable within the lumen of said tubular elongate member;
wherein:
a flow hole is defined radially through the wall of said tubular elongate member proximal to the distalmost end of said tubular elongate member; and
said anchor portion of said expandable member is coupled to the exterior of said tubular elongate member proximal to the flow hole whereby an inflation medium supplied into the lumen of said tubular elongate member flows radially out the flow hole to expand said anchor portion of said expandable member prior to expanding said invertible portion of said expandable member.

2. The system of claim 1, wherein said invertible portion of said expandable member is invertible into and within the lumen of said tubular elongate member when said expandable member is in an unexpanded configuration, and contact of said expandable member with the distalmost end of said tubular elongate member retains inflation medium within said anchor portion of said expandable member so that said anchor portion may expand prior to expansion of said invertible portion within and out of the distal end of said tubular elongate member.

3. The system of claim 1, wherein the flow hole is configured to assist with removal of inflation medium from said anchoring portion of said expandable member to fully deflate said expandable member.

4. The system of claim 1, further comprising a tether operably coupled with said invertible portion of said expandable member to assist with retracting said expandable member into the lumen of said tubular elongate member.

5. The system of claim 3, wherein said tether defines a lumen therethrough, said system further comprising an additional medical device insertable through the lumen through said tether.

6. The system of claim 5, wherein said additional medical device is a cauterization device.

7. The system of claim 5, wherein said additional medical device is an electrocauterization wire, said system further comprising a protective sleeve surrounding said electrocauterization wire to protect a portion of said invertible portion of said expandable member within the lumen of said tubular elongate member from said electrocauterization device.

8. The system of claim 1, further comprising an additional medical device extending along the exterior of said tubular elongate member or within an additional device lumen defined through said tubular elongate member separate from said lumen of said tubular elongate member.

9. The system of claim 8, wherein said additional medical device has a cauterization tip extendable distal to the distal end of said tubular elongate member.

10. The system of claim 1, further comprising a control handle operably coupled with the proximal end of said tubular elongate member, said control handle having an inflation port fluidly communicated with the lumen of said tubular elongate member and a fluid supply to supply inflation medium distally into the lumen of said tubular elongate member to extend radially through the flow hole to inflate said anchor portion of said expandable member prior to inflating said invertible portion of said expandable member.

11. A tissue-separating end effector of a device for separating tissue at a treatment site, said tissue-separating end effector comprising:
a tubular elongate member defined by a wall defining an interior of said tubular elongate member defining a lumen, and an exterior of said tubular elongate member, and extending between a proximal end and a distalmost end of a distal end of said tubular elongate member; and
an expandable member having an anchor portion extending along the exterior of the distal end of said tubular elongate member to an anchoring location proximal to the distalmost end of said tubular elongate member, and an invertible portion extendable within the lumen of said tubular elongate member;
wherein said anchor portion of said expandable member is inflatable to anchor said tissue-separating end effector with respect to the treatment site; and
said invertible portion is inflatable to extend distally out of the lumen of said tubular elongate member after inflation of said anchor portion.

12. The tissue-separating end effector of claim 11, wherein a flow hole is defined radially through the wall of said tubular elongate member proximal to the distalmost end of said tubular elongate member and fluidly communicated with said anchor portion of said expandable member.

13. The tissue-separating end effector of claim 12, wherein the anchoring location is proximal to the flow hole, whereby an inflation medium supplied into the lumen of the tubular elongate member flows radially out the flow hole to expand said anchor portion of said expandable member without expanding said invertible portion of said expandable member.

14. The tissue-separating end effector of claim 12, wherein the flow hole is configured to assist with removal of inflation medium from said anchoring portion of said expandable member to fully deflate said expandable member.

15. The tissue-separating end effector of claim 11, further comprising a tether operably coupled with said invertible portion of said expandable member to assist with retracting said expandable member into the lumen of said tubular elongate member.

16. A method of separating tissue at a treatment site, said method comprising:
extending a tissue-separating end effector to the treatment site, the tissue-separating end effector comprising a tubular elongate member defining a lumen therethrough, and an expandable member, the expandable member having an anchoring portion coupled to an anchoring location along exterior of the tubular elongate member proximal to a distalmost end of the tubular elongate member, and an invertible portion extendable within the lumen of the tubular elongate member;
expanding the anchoring portion of the expandable member prior to expanding the invertible portion of the expandable member to anchor the tissue-separating end effector with respect to the treatment site; and
after anchoring the tissue-separating end effector with respect to the treatment site, expanding the expandable member to evert distally out of the tubular elongate member to separate tissue.

17. The method of claim 16, further comprising inflating the anchoring portion with an inflation medium to expand the anchoring portion.

18. The method of claim 17, wherein a flow hole is defined radially through a wall defining the distal end of the tubular elongate member and proximal to the anchoring location, said method further comprising delivering an inflation medium into the lumen of the flexible elongate member to extend radially through the flow hole and into the anchoring portion of the expandable member to inflate the anchoring portion of the expandable member.

19. The method of claim 16, further comprising assisting eversion or inversion of the invertible portion of the expandable member by axial movement of a tether extending through the lumen of the tubular elongate member and operably coupled with the invertible portion of the expandable member.

20. The method of claim 19, further comprising extending an additional medical device through a lumen defined through the tether.
